(19) United States
(12) Patent Application Publication
(10) Pub. No.: US 2002/0136415 A1
(43) Pub. Date: Sep. 26, 2002

(54) Active noise cancellation for a vehicle induction system with selectable modelling noise

(75) Inventor: Paul D. Daly, Troy, MI (US)

Correspondence Address: LAURA M. SLENZAK, SIEMENS CORPORATION, 186 WOOD AVENUE SOUTH, ISELIN, NJ, 08830, US

(73) Assignee: Siemens VDO Automotive, Inc.

(21) Appl. No.: 10/061,589

(22) Filed: Feb. 1, 2002

Related U.S. Application Data
(60) Provisional application No. 60/277,194, filed on Mar. 20, 2001.

(51) Int. Cl. A61F 11/06; G10K 11/16; H03B 29/00

(52) U.S. Cl. 381/71.4; 381/86

(57) ABSTRACT

An active noise cancellation system is operative to reduce noise transmission through a vehicle air induction system. The active noise cancellation system includes a controller that utilizes computer modelling to ensure adequate noise cancellation. The controller generates a modelling sound to make determinations regarding the noise cancellation system, such as timing constraints associated with a speaker or microphone of the system. The modelling sound is selected to be consistent with a sound typically heard by an individual, given the current engine operating status. In another example, the selected sound masks another modeling sound such as random noise.

### CROSS REFERENCE TO RELATED APPLICATIONS

This application claims priority to U.S. Provisional Application Ser. No. 60/277,194, filed Mar. 20, 2001.

### BACKGROUND OF THE INVENTION

This invention generally relates to active noise cancellation in a vehicle induction system. More particularly, this invention relates to active noise cancellation using a selectable modelling noise.

Modern day vehicles typically include an air induction system. One drawback of air induction systems is that engine noise frequently travels through the air induction system and emanates out of the mouth of the air intake such that the noises are noticeable in the passenger compartment. Various efforts have been made to reduce the amount of engine noise traveling through the air induction system. Some arrangements include using passive devices such as expansion chambers and Helmholtz resonators. Other efforts include active methods such as anti-noise generators.

Typical active systems include a speaker that generates a sound to attenuate engine noise. The sound from the speaker is typically out of phase with the engine noise and combines with the engine noise such that the result is a reduced noise in the air induction system, which results in less noise transmission into the passenger compartment. The speaker sound can be referred to as a cancellation signal.

Cancellation signals typically are generated by digital signal processors such as microprocessors. The microprocessor typically requires some input from the engine environment to adequately address the need for noise cancellation. In some examples, computer modelling is used so that the microprocessor is able to provide a desired level of noise cancellation. One example aspect of the modeling is to compensate for delays between the speaker and a microphone that is part of the active noise cancellation system.

In one arrangement, the microprocessor drives the speaker so that a random noise or white noise is generated for a short time such as a few seconds. The microprocessor then processes input from the active noise cancellation system microphone and is able to determine time delays or other peculiarities of the particular system because the microprocessor has information regarding the timing, etc. of the generated noise.

One drawback associated with such an arrangement is that the randomly generated noise or white noise is often detected by a vehicle owner or another individual. Hearing such noises typically causes the individual concern as the noise is not recognized as an expected noise associated with the vehicle operation. In some situations the vehicle owner incorrectly believes that there may be a problem with the engine or another portion of the vehicle.

There is a need for an active noise cancellation system having modelling capabilities that utilizes an intentionally generated noise that will not cause a vehicle owner or another individual concern in the event the noise is heard by the individual. This invention addresses that need.

### SUMMARY OF THE INVENTION

In general terms, this invention is an active noise cancellation system for use on a vehicle that utilizes a selectable modelling noise that is chosen to be consistent with expected operating noises depending on the current state of vehicle operation.

One example system designed according to this invention includes a speaker situated to generate a noise into the vehicle air induction system. A microphone detects noises from within the vicinity of the speaker and the air induction system. A controller receives information from the microphone and drives the speaker to generate noises as needed. The controller determines a vehicle engine operation status and selects a noise to be generated by the speaker that is consistent with an expected noise given the current engine operation status. The controller is then able to use the generated noise to address any timing or other issues presented by the particular speaker and microphone arrangement.

In one example, the controller determines whether the engine is in a start up mode, idling or in a driving mode where the vehicle is being driven along a road surface, for example. If the controller determines that the engine is in the start up mode, the controller drives the speaker to generate a sound that is consistent with what an individual would hear while starting the vehicle engine. A variety of sounds or noises associated with vehicle start up can be used. If the controller determines that the engine is idling, the controller drives the speaker to generate a sound that is consistent with what an individual might hear while the engine is idling. One example idling sound includes the whirring noise associated with a cooling fan that may turn on while the engine idles. When the controller determines that the vehicle is being driven, a variety of sounds may be selected. One example such sound includes the noise that is heard when an air conditioning clutch engages or disengages. Other possible sounds when the vehicle is being driven include road noises, for example.

A method of generating a modelling noise in an active noise cancellation system according to this invention includes determining a status of the vehicle engine. Depending on the determined status, a modelling noise is selected to be consistent with an expected sound or noise that typically occurs when the engine is in the determined mode of operation.

In one example, the expected noise is generated to mask another sound used by the controller as part of the modeling. In this example, the expected noise effectively masks the random noise or other modeling sound.

The various features and advantages of this invention will become apparent to those skilled in the art from the following detailed description of the currently preferred embodiments. The drawings that accompany the detailed description can be briefly described as follows.

### BRIEF DESCRIPTION OF THE DRAWINGS

FIG. 1 schematically illustrates an active noise cancellation system designed according to this invention.

FIG. 2 is a flow chart diagram illustrating an example method of this invention.

### DETAILED DESCRIPTION OF THE PREFERRED EMBODIMENTS

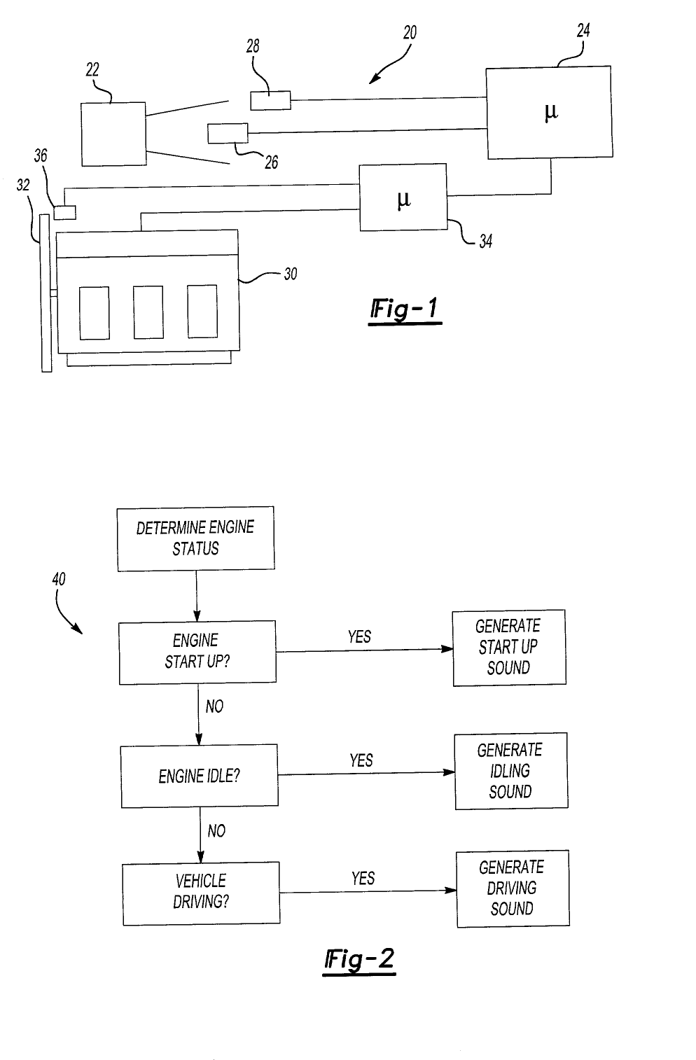

FIG. 1 schematically illustrates an active noise cancellation system 20 that is useful for reducing noises in a vehicle air induction system 22. The active noise cancellation system includes a controller 24 that may be a microprocessor, for example. The controller 24 drives a speaker 26 to generate noise cancellation sounds or signals that operate to cancel out engine noises otherwise propagated through the air induction system 22 in a known manner. The active noise cancellation system 20 also includes a microphone 28 through which the controller 24 gathers information regarding noises associated with the air induction system 22. The microphone 28 also provides feedback information to the controller 24 regarding operation of the speaker 26, for example.

The controller 24 preferably uses computer modelling such as C modelling to provide active noise cancellation. Such computer modelling techniques are known. The inventive arrangement differs from conventional techniques in that the noises intentionally generated during modelling are selected to be consistent with expected noises that may be heard by an individual depending on the current status of vehicle operation.

The air induction system 22 is associated with a vehicle engine 30. A conventional flywheel 32 is associated with the engine 30. The controller 24 gathers information regarding the current operating status of the engine 30 by communicating with a known engine controller 34, for example. Another source of information for the controller 24 is a conventional sensor 36 associated with the flywheel 32. In one example, the controller 24 communicates with the engine controller 34. In another example, the controller 24 gathers information directly from the flywheel sensor 36 or other sensors on the vehicle that provide information indicative of a current engine operating status. A pulse train from the sensor 36 provides information to the controller regarding engine operation.

Depending on the status of engine operation, in one example the controller 24 selects a modelling noise such that the sound generated by the speaker 26 during modelling is consistent with a sound or noise that an individual may hear under normal circumstances given the current engine operation status. As known, generating modelling sounds using a speaker allows an active noise cancellation system controller to accurately model the noise cancellation needed to effectively minimize noise propagation to the air induction system into the passenger compartment. When the controller 24 intentionally drives the speaker 26 to generate a modelling noise for a few seconds, the controller 24 can make determinations regarding the operation of the microphone 28 such as timing delays, etc., and this information can be used as part of the modelling technique. The controller is able to make calibration models of the received noise.

According to one example implementation of this invention, the controller 24 determines the current engine operating status. If the engine is in start up mode at the time that the controller 24 wishes to generate a modelling noise through the speaker 26, then the controller 24 selects a modelling noise or sound that is consistent with noises heard during engine start up. In this manner, the modelling noise is not detected by the vehicle owner or another individual as a strange or unexpected noise.

The decision strategy of this example is summarized in the flow chart 40 of FIG. 2.

When the controller 24 determines that the engine is idling, the controller 24 drives the speaker 26 such that the modelling noise mimics a sound heard during idling. One such example sound includes the whirring noise associated with a cooling fan. Because an individual may have heard such a noise in the past, when the modelling noise is generated to mimic that sound, the individual does not believe that anything unexpected has happened. Of course, other sounds associated with engine idle may be used.

When the controller 24 determines that the vehicle is being driven, the sound generated through the speaker 26 mimics the sound heard during normal driving. One example sound includes the noise of an air conditioning clutch engaging or disengaging. Other example sounds include road noises. Given this description, those skilled in the art will be able to select appropriate noises for a particular vehicle and the determined current engine operating status.

In another example, the controller 24 generates a random noise or white noise that is used as the modelling sound interpreted by the controller 24 through feedback received from the microphone 28, for example. The controller 24 also drives the speaker 26 to generate a masking sound that mimics a sound that would typically be heard by an individual given the current engine operating status. By masking the modelling noise, the modelling noise will not be detected by vehicle owner or individual, which avoids any concerns that may result if the modelling noise were heard and not understood by the individual to be associated with the active noise cancellation system.

The preceding description is exemplary rather than limiting in nature. Variations and modifications to the disclosed examples may become apparent to those skilled in the art that do not necessarily depart from the essence of this invention. The scope of legal protection given to this invention can only be determined by studying the following claims.

We claim:

1. An active noise cancellation system for use in a vehicle air induction system, comprising:
a speaker that selectively generates a sound;
a microphone situated to detect sounds from the speaker and sounds in the vicinity of the air induction system; and
a controller that selectively drives the speaker to generate a modelling sound used by the controller for modelling in the active noise cancellation system, the controller determining a current status of operation of the vehicle engine and selecting a modelling sound depending on the determined engine status.

2. The system of claim 1, wherein the controller determines whether the engine is in start up mode, idling, or driving.

3. The system of claim 2, wherein the modelling sound comprises a sound associated with starting up the engine when the controller determines that the engine is in start up mode.

4. The system of claim 2, wherein the modelling sound comprises the noise of a cooling fan when the controller determines that the engine is idling.

5. The system of claim 2, wherein the modelling sound comprises the noise of a clutch engagement when the controller determines that the engine is driving.

6. The system of claim 1, wherein the controller utilizes the modelling sound to make a determination regarding operation of the microphone.

7. The system of claim 1, wherein the controller 24 gathers information through the microphone based upon the modelling sound and responsively makes modeling calibration determinations.

8. The system of claim 1, including at least one sensor that provides an indication of engine operation and wherein the controller communicates with the sensor.

9. The system of claim 1, including an engine controller that provides an indication of engine operation and wherein the controller communicates with the engine controller.

10. A method of generating a modelling sound in an active noise cancellation system for use with a vehicle air induction system, comprising the steps of:
determining a current engine operating status; and
selecting a sound to use during modeling that is consistent with sound likely heard under conditions consistent with the current engine operating status.

11. The method of claim 10, including determining whether the engine is currently in start up mode, idling or driving.

12. The method of claim 11, including generating an engine start up sound when the engine is in start up mode.

13. The method of claim 11, including generating a sound that resembles a cooling fan noise when the engine is idling.

14. The method of claim 11, including generating a sound that resembles a clutch noise when the engine is driving.

15. The method of claim 10, including detecting the generated modelling noise and responsively making modelling calibration determinations.

16. The method of claim 10, including generating a modelling noise that comprises a random noise and generating the selected sound such that the sound masks the random noise such that the random noise can be detected within the active noise cancellation system but will not be heard by an individual.

17. The method of claim 16, including generating the random noise at a lower level than the selected sound.

18. The method of claim 16, including generating white noise as the random noise.

19. An active noise cancellation system for use in a vehicle air induction system, comprising:
a speaker that selectively generates a sound;
a microphone situated to detect sounds from the speaker and sounds in the vicinity of the air induction system; and
a controller that selectively drives the speaker to generate a modelling sound used by the controller for modelling in the active noise cancellation system, the controller determining a current status of operation of the vehicle engine and selecting a masking sound depending on the determined engine status.

20. The system of claim 19, wherein the controller drives the speaker to generate the masking sound at a higher level than the modeling sound.
